(19) United States
(12) Patent Application Publication
(10) Pub. No.: US 2024/0358533 A1
(43) Pub. Date: Oct. 31, 2024

(54) DEVICE AND METHOD FOR TREATING TISSUE USING A PATCH

(71) Applicants: BOSTON SCIENTIFIC MEDICAL DEVICE LIMITED, Galway (IE); BOSTON SCIENTIFIC SCIMED, INC., Maple Grove, MN (US)

(72) Inventors: Deepak Kumar SHARMA, Muzaffarnagar (IN); Sharath Kumar G, Kanakapura (IN); Kevin L. BAGLEY, Natick, MA (US); Paul SMITH, Smithfield, RI (US); Mazen YATIM, Southborough, MA (US); Samuel GOSHEN, Brookline, MA (US)

(21) Appl. No.: 18/626,986

(22) Filed: Apr. 4, 2024

Related U.S. Application Data
(60) Provisional application No. 63/498,636, filed on Apr. 27, 2023.

(51) Int. Cl. A61F 2/958; A61B 17/00; A61F 2/95

(52) U.S. Cl. CPC A61F 2/958; A61B 17/00234; A61F 2/9522; A61B 2017/0034

(57) ABSTRACT

A device includes a flexible sheath, a member, an expansion device, and a therapeutic patch. The sheath has a length selected to extend from a distal end which, when the device is in operative configuration, is adjacent to the target site, to a proximal end. The member extends through the sheath from a proximal end to a distal end. The expansion device is coupled to and extends distally from a distal end of the member. The patch is wrapped around the expansion device and forced radially outward into contact with and adhere to a first target portion of tissue when the expansion device is moved to deployed configuration. The expansion device is movable, after the patch has been adhered to the portion as desired, from deployed configuration to insertion configuration to separate the patch from the expansion device leaving the patch in a desired position adhered to the portion.

### PRIORITY CLAIM

The present disclosure claims priority to U.S. Provisional Patent Application Ser. No. 63/498,636 filed Apr. 27, 2023; the disclosure of which is incorporated herewith by reference.

### FIELD

The present disclosure relates to a minimally invasive delivery of a patch to cover a large portion of target tissue to achieve, for example, hemostasis. More specifically, the present disclosure relates to the intraluminal delivery of a patch positioned over an expandable body so that, upon reaching the target site, the patch may be moved radially outward to contact, adhere to and cover a large area of target tissue to be treated.

### BACKGROUND

The treatment of bleeding ulcers generally involves injection therapy, thermal therapy, and mechanical therapies which currently impose significant limits as these treatments are generally expensive, time intensive and limited in the surface area of tissue that can be treated. The only solution generally available for chronic ulcer management is gastric bypass which has various limitations, costs and drawbacks of its own.

### SUMMARY

The present disclosure relates to a device for treating tissue. The device includes a hollow sheath having sufficient flexibility to be inserted to a target site within a living body within a flexible endoscope. The sheath has a length selected to extend from a distal end which, when the device is in an operative configuration, is adjacent to the target site, to a proximal end which remains outside the living body accessible to a user;

The device also includes a flexible hollow member extending through the sheath from a proximal end that remains outside the living body to a distal end which may be extended distally out of the sheath to contact a target portion of tissue adjacent to the target site. In addition, the device includes an expansion device coupled to and extending distally from a distal end of the hollow member, the expansion device being movable from a radially compressed insertion configuration to a radially expanded deployed configuration.

Furthermore, the device includes a first therapeutic patch wrapped around the expansion device. The first patch is configured to be forced radially outward into contact with and adhere to a first target portion of tissue when the expansion device is moved to the deployed configuration, the expansion device being configured to be moved, after the first patch has been adhered to the first target portion of tissue as desired, from the deployed configuration to the insertion configuration to separate the first patch from the expansion device leaving the first patch in a desired position adhered to the first target portion of tissue.

In an embodiment, the expansion device comprises a plurality of members extending from a proximal end of the expansion device to a distal end of the expansion device. In addition, the device further includes a control wire coupled to a distal end of the expansion device, movement of the control wire proximally relative to the expansion device compressing the expansion device along a longitudinal axis thereof and expanding the expansion device radially away from the longitudinal axis.

In an embodiment, the members of the expansion device are formed as an expandable tubular stent.

In an embodiment, a first member of the expansion device includes a proximal flexible section coupled to the hollow member, a rigid central member extending distally from the proximal flexible section and a distal flexible section coupled to a distal end of the control wire, the control wire extending proximally through the expansion device and through the hollow member to a proximal end that remains outside the living body manipulable by a user of the device.

In an embodiment, the device further includes a control member coupled to a slider of a handle of the device. A proximal end of the hollow member is coupled to the handle. The control member extends through the hollow member and the expansion device to couple to a distal end of the expansion device.

In an embodiment, the proximal and distal flexible sections of the first member include one of Nitinol, of Nitional cobalt alloy wire, pebax, nylon, ptfe and copper.

In an embodiment, the rigid central member includes one of stainless steel, hypotube, a biocompatible hard plastic.

In an embodiment, the device further includes a second therapeutic patch wrapped around the expansion device and radially outside the first therapeutic patch, the second patch being configured to be forced radially outward into contact with and adhere to a second target portion of tissue when the expansion device is moved to the deployed configuration, the expansion device being configured to be moved, after the second patch has been adhered to the second target portion of tissue as desired, from the deployed configuration to the insertion configuration to separate the second patch from the expansion device leaving the second patch in a desired position adhered to the second target portion of tissue.

In an embodiment, the first therapeutic patch has a width extending along a longitudinal axis of the expansion device and a length transverse to the width. The first therapeutic patch is wrapped around the expansion device so that the length of the first therapeutic patch extends circumferentially around the expansion device. The length of the first therapeutic patch is configured to be at least as great as a diameter of the expansion device in the deployed configuration.

In an embodiment, the first therapeutic patch includes an adhesive having a predetermined cure time and wherein the first therapeutic patch is configured to be removable from tissue against which it has been pressed before the predetermined cure time has elapsed since the first therapeutic patch was pressed against the tissue.

In addition, the present disclosure relates to a method for treating tissue. The method includes inserting into a living body via a working channel of a flexible endoscope a device including a hollow sheath having received therein an expansion device with a first therapeutic patch wrapped around the expansion device, wherein the sheath is maintained during insertion in an insertion configuration in which the expansion device and the first therapeutic patch are maintained within the hollow sheath; extending the sheath distally out of the endoscope to a target site adjacent to target tissue to be treated; moving the sheath relative to the expansion device and the first therapeutic patch to expose the expansion device and the first therapeutic patch distally of a distal end of the sheath; expanding the expansion device to move the first therapeutic patch radially outward into contact with a first target portion of tissue to be treated; and radially contracting the expansion device out of contact with the first therapeutic patch to leave the first therapeutic patch in a desired position adhered to the first target portion of tissue.

In an embodiment, the first therapeutic patch has a width extending along a longitudinal axis of the expansion device and a length transverse to the width. The first therapeutic patch is wrapped around the expansion device so that the length of the first therapeutic patch extends circumferentially around the expansion device. The length of the first therapeutic patch is configured to be at least as great as a diameter of the expansion device in the deployed configuration. The method further comprising the step of pressing the first therapeutic patch against the first target portion of tissue and moving the expansion device relative to the first target portion of tissue in a direction corresponding to the length of the first therapeutic patch to unroll the first therapeutic patch from the expansion device over the first target portion of tissue until the entire length of the first therapeutic patch has been adhered to the first target portion of tissue.

In an embodiment, the device further includes a second a therapeutic patch wrapped around the first therapeutic patch and the expansion device.

In an embodiment, the expansion device is a radially expandable stent and therein the device includes a mechanism for longitudinally compressing the stent to radially expand the stent.

In an embodiment, the expansion device is coupled to a distal end of a hollow member that extends through the sheath to a proximal end that remains outside the living body, the expansion device including a plurality of longitudinal members, each of the longitudinal members including a proximal flexible section coupled to the hollow member, a rigid central member extending distally from the proximal flexible section and a distal flexible section, further comprising the step of, manipulating a control wire that extends through the hollow member and the expansion device to couple to the distal flexible sections of the longitudinal members to longitudinally compress and radially expand the expansion device.

### BRIEF DESCRIPTION OF DRAWINGS

FIG. 1a shows a perspective view of a device according to an exemplary embodiment.

FIG. 1b shows a distal end of the device of FIG. 1a.

FIG. 1c shows a proximal end including a handle of the device of FIG. 1a.

FIG. 2 shows a perspective view of a handle of the device according to the exemplary embodiment of FIG. 1a.

FIG. 3 shows a perspective view of the handle of FIG. 1a with the sheath pulled proximally into a deployed configuration.

FIG. 4 shows a perspective view of distal end of the device of the embodiment of FIG. 1a with the therapeutic patch exposed in the deployed configuration.

FIG. 5 shows a side view of a stent of the embodiment of FIG. 1a without the patch wrapped therearound.

FIG. 6 shows a side view of a distal end of device with the sheath removed therefrom.

FIG. 7 shows a side view of the distal end of the device of FIG. 1a with the sheath partially withdrawn proximally from the patch and stent in a position between the insertion and deployed configurations.

FIG. 8 shows a perspective view of the distal end of the device of FIG. 1a with the sheath withdrawn proximally from the patch and the stent.

FIGS. 9a-9b show the stent in an expanded state and the distal end of the device of FIG. 1a.

FIG. 10a shows a perspective view of the distal end of the patch of the device of FIG. 1a without the stent received therein.

FIG. 10b shows an expanded perspective view of a portion of the distal end of the patch of FIG. 10a;

FIG. 11 shows a perspective view of the distal end of the patch of FIGS. 10a-b with the turns of the patch separated from one another to show the multiple circumferential wrappings of the patch about the stent.

FIG. 12 shows a perspective view of a patch assembly according to an alternate embodiment in which multiple patches are circumferentially wrapped around the stent sequentially.

FIGS. 13a-b show a perspective view of a distal end and a proximal end of a device according to a further embodiment.

FIG. 14 shows the expansion apparatus of the device of FIGS. 13a-b with the patch removed therefrom.

FIG. 15 shows a perspective view of the distal end of the expansion apparatus of the device of FIGS. 13a-b with the patch removed.

FIG. 16 shows a perspective view of the distal end of the expansion apparatus of the device of FIGS. 13a-b with the patch wrapped thereover.

FIG. 17 shows a perspective view of the proximal end of the expansion apparatus of FIGS. 13a-b.

FIGS. 18a-b show a perspective view of the expansion apparatus in the expanded state and the proximal end of the device of FIGS. 13a-b with the expansion apparatus.

### DETAILED DESCRIPTION

The embodiments described herein relate to apparatus and methods for the minimally invasive application of a patch of material to tissue within a living body. Although specific embodiments will be described in regard to the endoscopic application of a patch or patches of material to treat tissue within the digestive tract (e.g., esophagus, stomach, small intestine and colon, common bile duct and pancreatic ducts), those skilled in the art will understand that the system apparatus and techniques may be employed to apply a patch or patches of material to tissue within any other internal bodily tissues. Furthermore, as used in this application the terms distal and proximal refer to directions along a device from parts closer to a user (proximal) and further (distal) from the user. In general, the proximal end of such a device will include a handle which, during use of the device, remains outside the body accessible to the user and an end effector at the distal end which is inserted into the body (e.g., via a naturally occurring bodily orifice and navigated to a target site adjacent to target tissue to be treated via a natural body lumen).

However, those skilled in the art will understand that other tissue that may be accessed within the body via other pathways (e.g., along a path that proceeds through a natural body lumen to a point at which the device is passed out of the lumen (e.g., via a surgical opening formed therein) and that this tissue may then be treated by the application of a patch in the same manner described for the application of a patch to tissue on the inner wall of the lumen. Furthermore, embodiments will be described in which a therapeutic patch is applied to target tissue to achieve or assist in achieving hemostasis. However, as those skilled in the art will understand, such a therapeutic patch may be applied using the same apparatus and techniques where the patch is configured to treat conditions different from or the bleeding/tissue opening sealing function of the described embodiments.

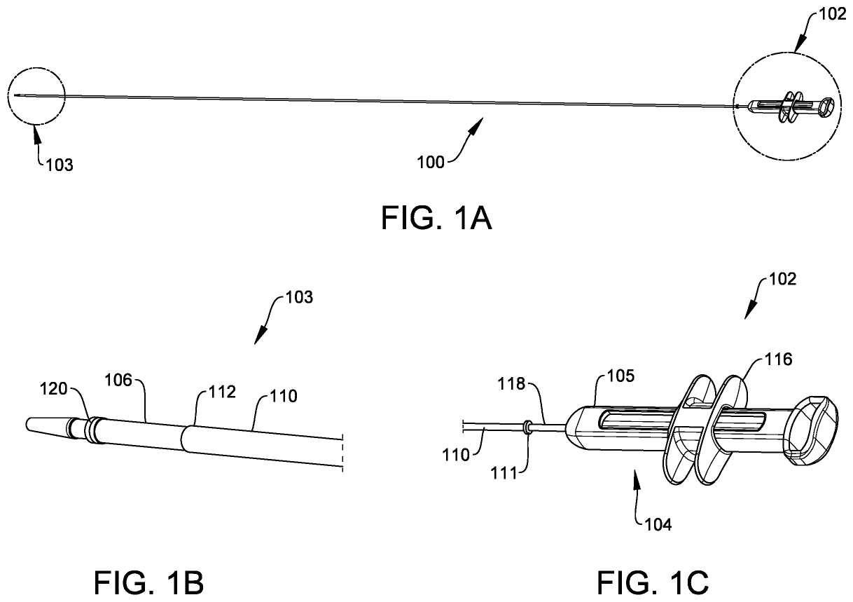

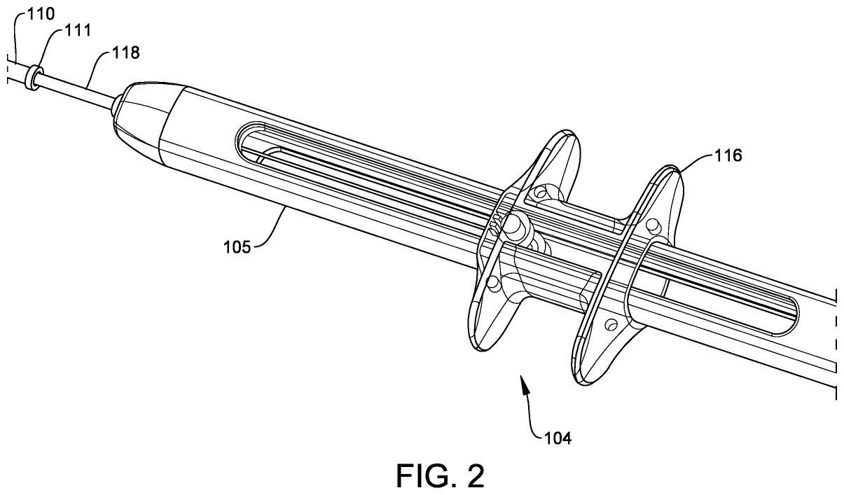

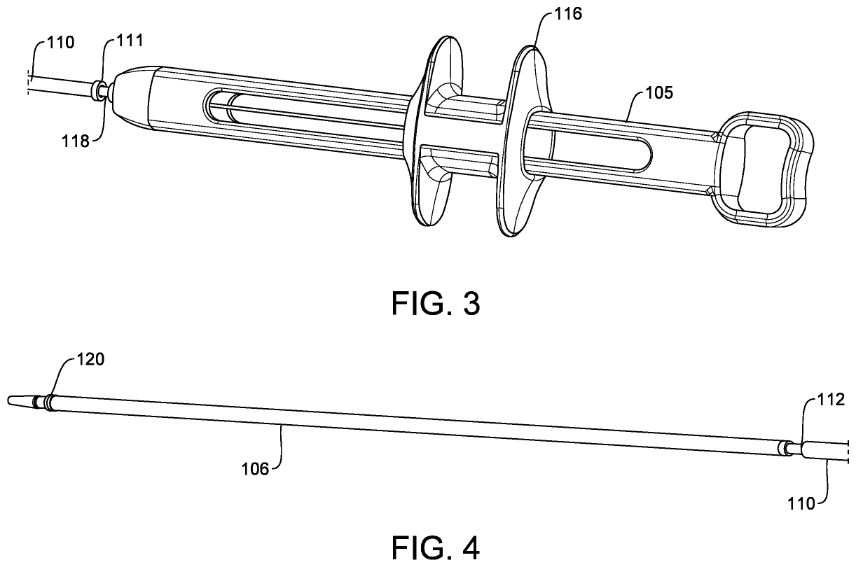

FIGS. 1-6 show a device 100 extending from a proximal end 102 including a handle 104 to a distal end 103 including a therapeutic patch 106 that is wrapped around an expanding element (e.g., stent 108). A hollow sheath 110 extends distally from a proximal end 111 to a distal end 112 with a length of the sheath 110 being selected to be movable to an insertion configuration (i.e., a first position) in which the distal end 112 covers the distal end of the stent 108 and the patch 106 while the proximal end 111 of the sheath 110 extends out of a handle of the endoscope accessible to a user.

A proximal end of a control wire 114 is coupled to a slider 116 of the handle 104 and the control wire 114 extends distally through the handle 104 to pass into a tube 118. The tube 118 is slidably received within the sheath 110 and the proximal end of the stent 108 is coupled to the tube 118 with the stent 108 extending distally from the distal end of the tube 118. The proximal end of the tube 118 is coupled to the distal end of the handle body 105. The patch 106 is wrapped around the stent 108 as will be described in more detail below.

As indicated above, the length of the sheath 110 and length of the tube 118 plus the length of the stent 108 that extends distally therefrom are selected so that, in an insertion configuration, the stent 108 and the patch 106 wrapped therearound form a cylinder that is maintained within the sheath 110 out of contact with the bodily fluids that surround the sheath 110. Thus, those skilled in the art will understand that, due to size constraints and to limit the entry of bodily fluids into the sheath 110 before the patch 106 is extended distally out of the sheath 110, the outer diameter of the cylinder formed by the patch 106 wrapped around the stent 108 is, in an exemplary embodiment selected to closely match the inner diameter of the sheath 110.

The tube 118 is hollow and surrounds the control wire 114 so that the control wire 114 passes through the tube 118 and through the stent 108 to couple to a ring 120 at a distal end 108x of the stent 108. Thus, movement of the control wire 114 proximally and distally within the sheath 110 (via movement of the slider 116 relative to a body 105 of the handle 104) moves the distal end of the stent 108 proximally and distally within the sheath 110 and moves the distal end of the stent 108 proximally and distally relative to the tube 118.

Thus, to place the device 100 in the insertion configuration (with the stent 108 and the patch 106 exposed distally of the distal end 112 of the sheath 110), the sheath 110 is slid distally over the tube 118 until the entirety of the stent 108 and the patch 106 (or alternatively, at least the distal end of the patch 106) is received within the sheath 110. As would be understood by those skilled in the art, in the insertion configuration, the slider 116 is retracted to a proximal-most position with respect to the body 105 of the handle 104 so that the stent 108 and the patch 106 are radially compressed to fit within the lumen of the sheath 110. In this configuration, the sheath 110 may be inserted through the working channel of an endoscope (not shown) that has been advanced to a target site adjacent to target tissue within the body that is to be treated via application of the patch 106.

Alternatively, the device 100 may be inserted into the endoscope before the endoscope is inserted into the body and advanced to the target site within the endoscope. The user may then advance the sheath 110 distally out of the working channel (e.g., into the body lumen toward the target tissue) by pushing the proximal end 111 distally into the endoscope while holding the handle 104 in position. The user may perform this process under visual observation via a vision system of the endoscope as would be understood by those skilled in the art.

When the distal end 112 of the sheath 110 is positioned relative to the target tissue as desired, the user then draws the sheath 110 proximally over the tube 118 (e.g., until the proximal end of the sheath 110 contacts the body 105 of the handle 104) by pulling the proximal end 111 of the sheath 110 proximally toward the handle 104 to place the device 100 in the deployed configuration. This draws the distal end 112 of the sheath 110 proximally over the stent 108 and the patch 106 to expose the stent 108 and the patch 106 distally of the distal end 112 of the sheath 110.

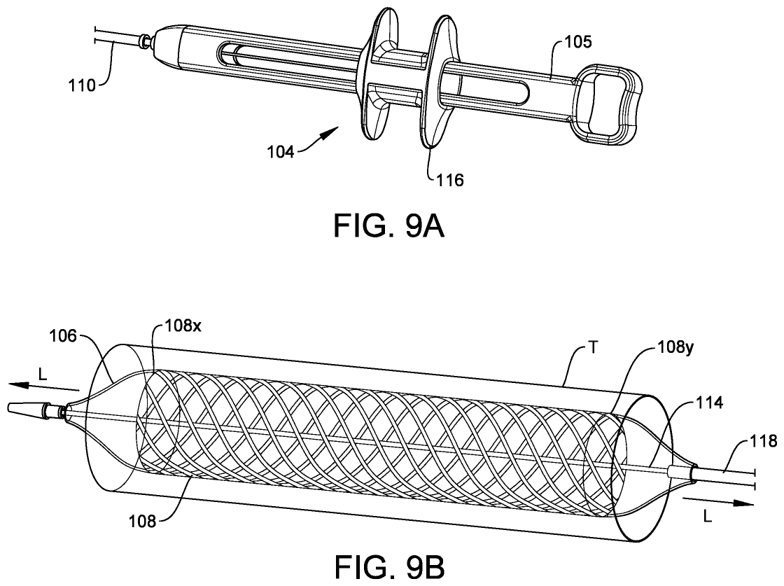

As would be understood by those skilled in the art, when the stent 108 moves out of the constraint of the sheath 110, the stent 108 may begin to expand (e.g., through any number of mechanisms such as a bias imparted to the members forming the stent, a memorized shape imparted to a shape memory material such as Nitinol, etc.). This will begin to expand the patch 106 radially outward toward the target tissue. As would be understood by those skilled in the art, this self-expansion of the stent 108 may, in certain circumstances, be sufficient to move an outer surface of the patch 106 radially outward into contact with the target tissue. Specifically, as shown in FIGS. 9a-b, when the target tissue forms part of the wall of a tubular organ T (e.g., esophagus, colon, small intestine), the outward pressure applied by the stent 108 urging the patch 106 into contact with the target tissue may be sufficient to adhere the patch 106 to the target tissue. The requirements for achieving a desired adherence of the patch 106 to target tissue and the characteristics of the patch 106 and the expansion apparatus employed to achieve this adherence will be described in more detail below.

As indicated above, the ring 120 at the distal end 108x of the stent 108 is coupled to the distal end of the control wire 114. Thus, after the stent 108 and the patch 106 have been distally extended out of the sheath 110, the user may draw the slider 116 proximally relative to the body 105 of the handle 104 to pull the distal end of the control wire 114 proximally relative to the sheath 110. Drawing the distal end 108x of the stent 108 proximally while the proximal end 108y of the stent 108 is prevented from moving proximally by the tube 118 compresses the stent 108 longitudinally (i.e., the distance between the distal end and the proximal end 122 of the stent 108 is reduced along the axis L) causing the stent 108 to expand radially outward. This drives the patch 106 that is wrapped around the stent 108 to expand radially as well.

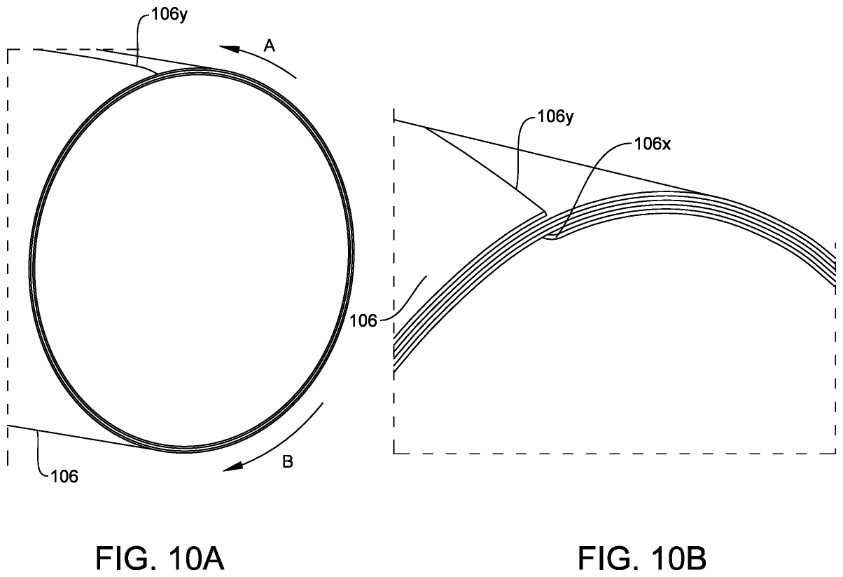

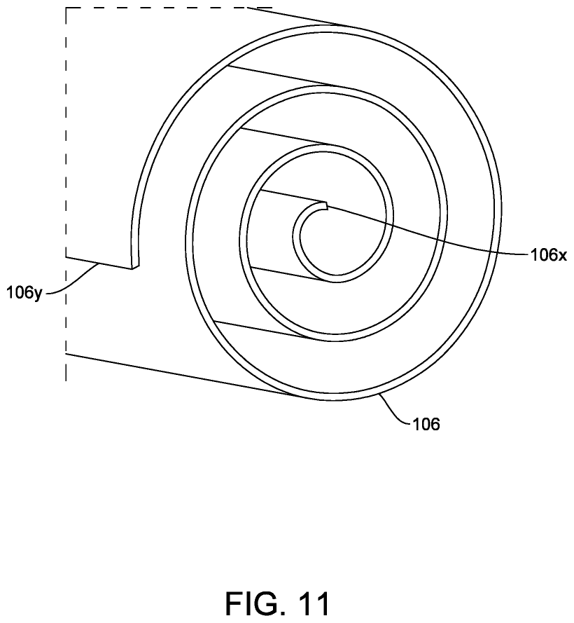

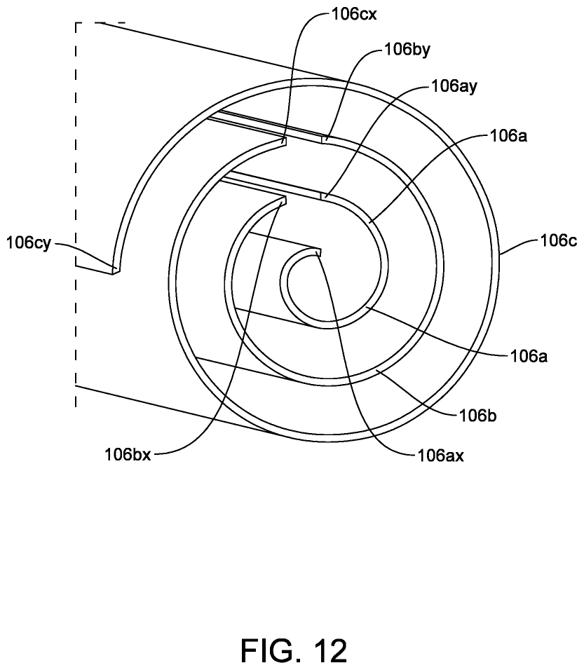

Those skilled in the art will understand that the patch 106 is wrapped around the stent 108 loosely so that it can freely unwind as the stent 108 expands—i.e., so that the patch 106 loosens instead of ripping or impeding expansion of the stent 108. In this embodiment, the patch 106 is wrapped around the stent 108 for more than a single circumference of the stent 108 so that as the patch 106 is expanded from its initial state in the insertion configuration the patch 106 continues to cover the entire circumference of the stent 108. That is, as shown in FIGS. 10a-b, for example, the patch 106 is wrapped more than 360 degrees around the stent 108 to permit for the radial expansion of the patch 106 which permits the material of the patch 106 to maintain full circumferential coverage of the stent 108 as the radius of the stent 108 (and the circumference of the stent 108) expands. More specifically, the patch 106 of FIGS. 10a-b extends from a first free end 106x resting on the surface of the stent 108 to a second free end 106y.

As would be understood by those skilled in the art, this type of mechanical longitudinal compression of the stent 108 can apply significant radially outward pressure as the longitudinal force exertable by a user via the slider 116 is considerable. For example, using the slider 116, a user may generate an outward radial force of from 1 to 15 lbf which, as those skilled in the art will understand, corresponds, due to known mechanical advantage, to a radially outward force of 5-20 lbf applied by expansion of the stent 108 to the patch 106 and the surrounding tissue.

As would be understood by those skilled in the art, for devices that are to be inserted to target sites via the working channel of an endoscope the outer diameter of the sheath 110 is limited by the inner diameter of the working channel of the endoscope being used. For example, for an endoscope having an outer diameter of 10.5-12 mm, the working channel generally has an inner diameter of 2.8 mm or less. For such an endoscope, the sheath 110 will generally have an outer diameter of approximately 2.7 mm and an inner diameter of 2.6 mm. The outer diameter of the patch 106 that is wrapped around the stent 108 within the sheath 110 will generally be between 2.5 mm and 0.6 mm. In this case, an outer diameter of the stent 108 when in the insertion configuration will be approximately 1 mm to 2.5 mm. As would be understood by those skilled in the art, for a stent 108 of this size, the patch 106 may be radially expanded to a diameter of 50 mm. Thus, for body lumens having a circumference of 50 mm or less, the patch 106 may be radially expanded via this longitudinal compression of the stent 108 alone into contact with tissue extending around the entire circumference of the body lumen. That is, radially expanding the stent 108 to its maximum extent will urge the patch 106 outward to fully open the body lumen so that tissue around the entire circumference of the lumen is forced into contact with and adhered to the patch 106.

A technique for applying a patch 106 to larger circumferential portions of tissue or to partially circumferential portions of tissue in large diameter spaces (spaces having a diameter beyond the maximum radial expansion of the patch 106 achievable by manipulation of the stent 108) will be described in more detail below. Those skilled in the art will understand that all of the dimensions given here are illustrative and if, for example, an endoscope or working channel of an endoscope presents other dimensions and/or anatomical considerations present challenges other dimensions may be employed as necessary.

As would be understood by those skilled in the art the patch 106 may be a biocompatible, flexible and resorbable gelatin patch configured to begin a coagulation cascade via mechanical interaction. For example, in one embodiment, the patch 106 is formed of one or more Chitosan sheaths. As would be understood by those skilled in the art, Chitin and its deacetylated derivative, chitosan, are a family of linear polysaccharides including varying amounts of (β1→4) linked residues of N-acetyl-2 amino-2-deoxy-D-glucose (glucosamine, GlcN) and 2-amino-2-deoxy-D-glucose (N-acetyl-glucosamine, GlcNAc) residues. The size of the patch 106 may vary depending on the application and the target anatomy. As an example, the patch 106 may have a size of, for example, 10″×10″ but may have any size up to 20″×20″. The patch 106 may be used, for example, to stop the bleeding/cover bleeding ulcers even where larger surface areas of target tissue need to be treated.

When the patch 106 is formed of Chitosan, the patch 106 may be repositioned. That is, if after the patch 106 has been initially placed on target tissue, the user believes the position is not correct (e.g., based on observation via the endoscopic vision system), the user may remove the patch 106 from the target tissue before adhesive in the patch 106 has cured. That is, after the patch 106 is pushed against the tissue, adhesive in the patch 106 takes a certain amount of time to adhere to the tissue (i.e., a known curing time must elapse when the patch 106 is in contact with body fluid and tissue before the patch 106 is adhered thereto).

Thus, if the user sees that the patch 106 (e.g., under direct visualization via the scope vision system) has been placed in contact with tissue in a position that is not correct, the user may can retract the stent radially inward (by pushing the slider 116 distally relative to the handle body 105) to extend the control wire 114 distally drawing the stent 108 and the patch 106 radially inward out of contact with the tissue. As the stent 108 retracts radially inward, the patch 106 rolls back on the stent 108 due to its stiffness. The user then repositions the stent 108 and the patch 106 by moving the sheath 110 and/or by altering the aim of the distal end of the endoscope until the proper position of the patch 106 relative tissue has been achieved. The user may then reapply the patch 106 in the same manner described above.

In a similar manner, a user may use the same stent 108 to place multiple patches 106 over different target portions of tissue at different locations. For example, as shown in FIG. 12, instead of a single patch 106 rolled around the stent 108 as a single continuous sheet, any number of patches (e.g., a first patch 106a, a second patch 106b, a third patch 106c) can be rolled around the stent 108 and separately placed on different portions of tissue. In such an embodiment, the first patch 106a is rolled around the stent 108 from a first end 106ax to a free end 106ay and the second patch 106b (either not connected to the first patch 106a or releasably coupled thereto) is wrapped around the stent 108 in the same direction as the first patch 106a starting from a first end 106bx adjacent to the free end 106ay of the first patch 106a so that the second patch 106b covers the first patch 106a.

Subsequent patches 106n are then wrapped further around the stent 108 with each subsequent patch 106 starting adjacent to the exposed end of the previous patch 106 and laying over the top of this immediately previous patch 106 and so on. To place these individual patches 106a-n, a user positions the stent 108 and the patches 106a-n as desired relative to a first target portion of tissue to be treated. The user expands the stent 108 as described above to expand an outermost one of the patches (e.g., the third patch 106c) in this embodiment) into contact with this first target portion of tissue and, when the third patch 106c has properly adhered to the first target portion of tissue as desired, the user extends the control wire 114 distally to radially retract the stent 108 until the second patch 106b immediately radially within the third patch 106c is moved out of contact with the third patch 106c. The user then positions the second patch 106b in a desired location relative to a second portion of tissue to be treated and adheres the second patch 106b to the second portion of tissue in the same manner described above for the third patch 106c. The process is repeated in the same manner for the first patch 106a (or until the desired number of patches 106 have been placed as desired).

Alternatively, the patch 106 may be an Electrospun patch including nano fibers or formed of a biocompatible material such as nylon, urethane, nylon or latex) capable of forming a flexible collapsible member, or membrane as would be understood by those skilled in the art. The patch 106 may also include any combination of these materials as well as any other bioadhesive materials such as chitosan, modified chitosan, cellulose, pHEMA, PVA, PEG, or composites of these polymers. The patch may alternatively be formed as a mesh of Polypropylene, Polyester and/or ePTFE. In addition, fibrin glue or any other suitable adherent may be employed on the outer layer of patch. As the fibrin glue comes in contact with the tissue, it helps to attach the patch to the tissue.

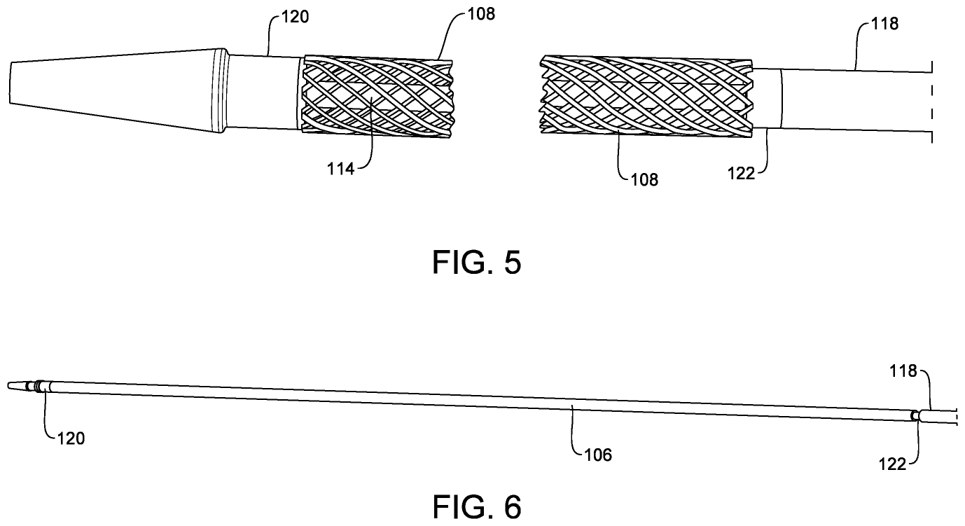

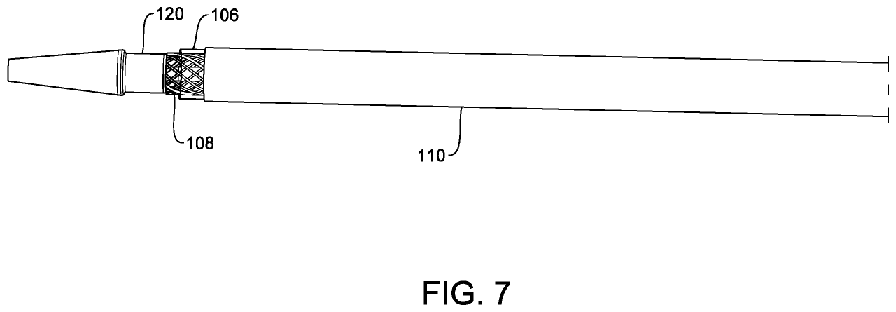

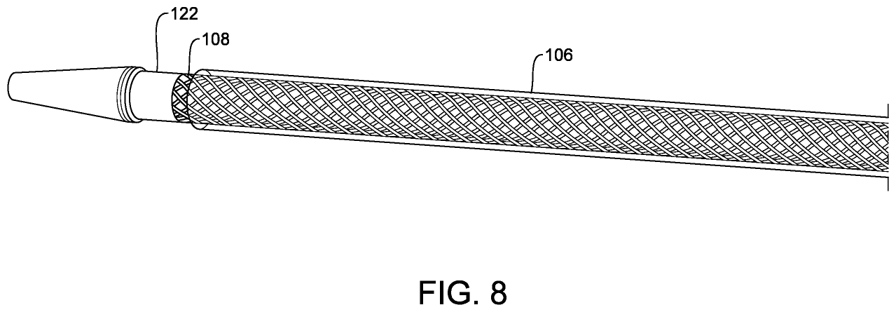

As would be understood by those skilled in the art, the stent 108 may be formed as a wire mesh configured to be transitioned from a collapsed state in which the outer diameter of the stent 108 is reduced to a size that is slidable within the lumen of the sheath 110 and an expanded state (shown in FIG. 5) in which the stent 108 is radially expanded (e.g., by longitudinally compressing the stent 108 (reducing its length along a proximal-distal axis). However, those skilled in the art will understand that any of a variety of other expansion mechanisms may be employed depending on the details of the procedure, the construction of the patch 106 to be applied, the type of tissue to which the patch 106 is to be adhered, forces to be expected to be applied to the patch 106 after deployment (e.g., through patient movements or through internal bodily processes impacting the activities of surrounding tissue, etc.).

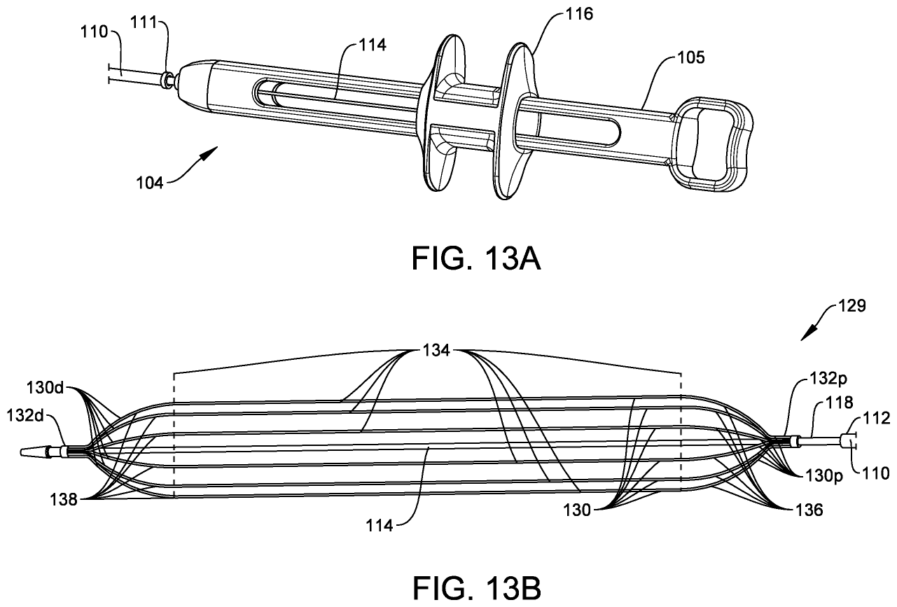

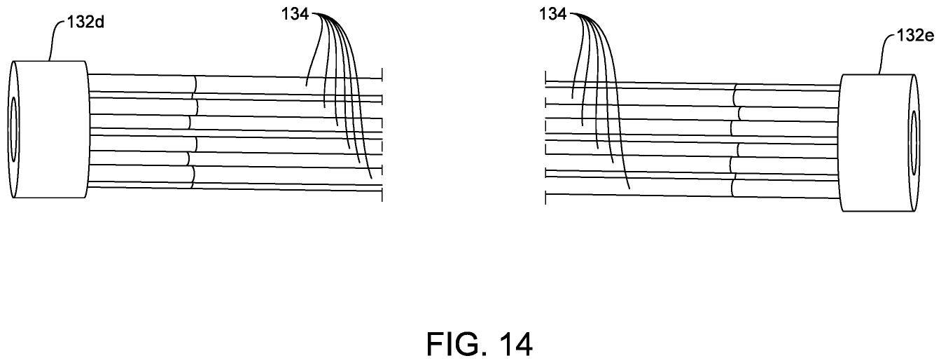

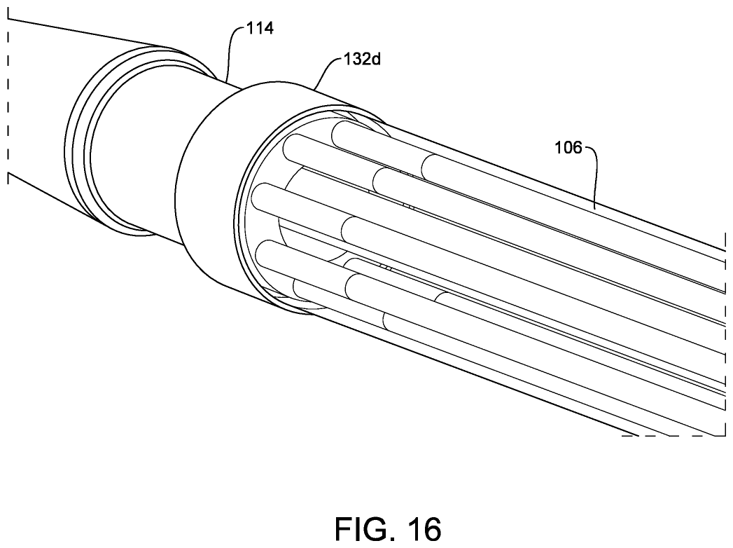

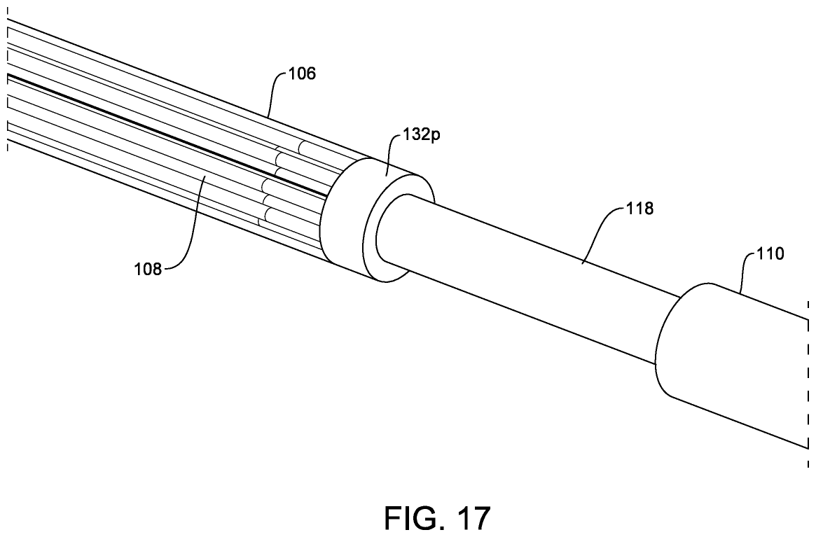

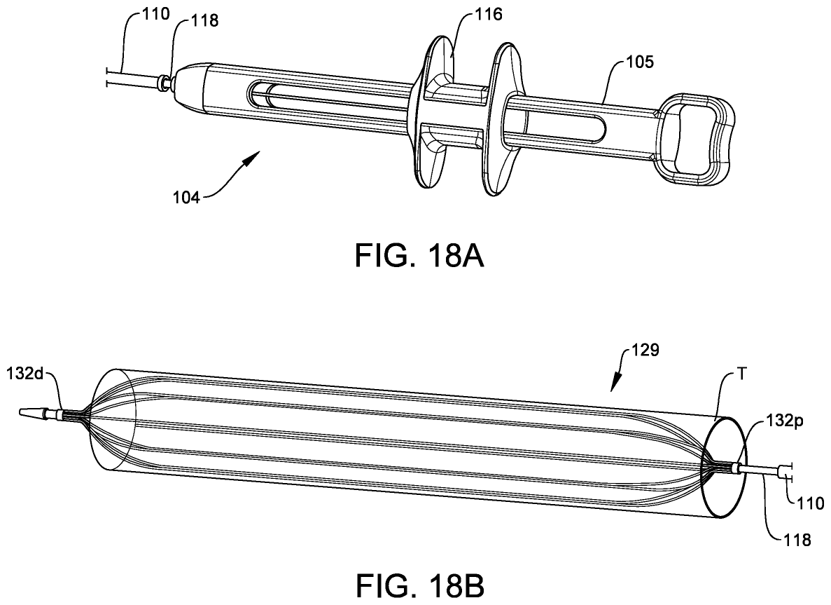

As shown in FIGS. 13-18, an expansion device 129 according to a further embodiment is formed as a series of longitudinal members 130. Each of the longitudinal members 130 extends from a proximal end 130p to a distal end 130d. All of the proximal ends 130p are coupled to one another via a proximal ring 132p and the distal ends 130d of the longitudinal members 130 are attached to one another via a distal ring 132d. Each of the longitudinal members 130 of this embodiment is formed of a substantially rigid linear section 134 coupled to at its proximal end to a corresponding flexible proximal portion 136. The distal end of each of the linear sections 134 is coupled to a corresponding flexible distal portion 138.

In an exemplary embodiment, the flexible proximal and distal portion 136, 138, respectively, may be formed of Nitinol while the linear sections are formed of stainless steel or from portions of a hypotube. Alternatively, the flexible portions may be formed of any one or a combination of Nitional cobalt alloy wire, plastics made of pebax, nylon, ptfe or copper, etc. and the stiff portion may alternatively be formed of any of a number of biocompatible hard plastics such as ABS.

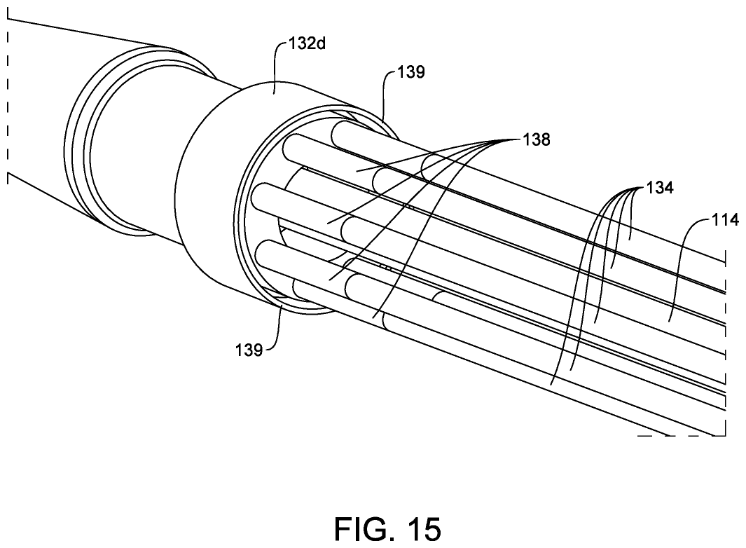

As seen in FIG. 15, a proximal surface of the distal ring 132d includes a slot 139 extending distally into a radially outer portion of a proximal side of the distal ring 132d so that the patch 106 can be inserted therein and the patch 106 will lie flush with the radially outer surface of the distal ring 132d when the patch 106 is wrapped around the expansion device 129. As would be understood by those skilled in the art, a similarly sized, shaped and positioned slot may be formed at a radially outer edge of the distal end of the proximal ring 132p so that the patch 106, when wrapped about the expansion device 129 does not increase the outer diameter of the expansion device 129 beyond the limit already established by the radially outer-most surfaces of the proximal and distal rings 132p, 132d, respectively.

In use, the user will pass the sheath 110 including the tube 118, the stent 108 and the patch 106 distally into the working channel of an endoscope with the stent 108 and the patch 106 radially compressed and in the insertion configuration received inside the sheath 110. The user may then advance the endoscope to a target site within the body using any convention technique (e.g., using visual feedback via the endoscopic vision system to determine when the distal end of the endoscope is positioned as desired relative to a target portion of tissue to be treated).

The user may then extend the sheath 110 distally out of the distal end of the endoscope (i.e., to project distally out of a distal end of the working channel). At this point the user may extend the patch 106 and the stent 108 out of the sheath 110 by moving the slider 116 distally relative to the handle body 105. The user may then confirm that the patch 106 is positioned as desired relative to the target tissue. For example, the user may wish to confirm that a distal end of the patch 106 is extended past a distal end of the target portion of tissue while the proximal end of the patch 106 is proximal of a proximal end of the target portion of tissue.

If the target tissue is a portion of a body lumen of less than a predetermined maximum diameter (e.g., the diameter to which the expansion device may radially expand the patch 106 to press against the target tissue with a desired force (e.g., a force sufficient to ensure a desired adherence between the patch 106 and the target tissue), the user may then expand the expansion device (e.g., the stent 108) to force the patch 106 radially outward into contact with a fully circumferential portion of the tissue extending proximally and distally beyond the ends of the target portion of tissue so that the entire portion of target tissue will be covered by the patch 106.

If the portion of tissue to be treated is not in a vessel having a lumen of a size admitting of full circumferential application of the patch 106 via expansion of the expansion device alone (e.g., if the target portion of tissue is a part of a wall of a larger vessel such as the stomach), the user, after expansion of the expansion device, will manipulate the endoscope to press a first portion of the patch 106 against the wall of the tissue. The user would locate a free end of the patch 106 (e.g., by visually identifying a marker formed on the patch 106 at the free edge and place this free edge in contact with the tissue and unroll the patch 106 in a direction opposite the direction in which the patch 106 was rolled onto the expansion device (e.g., stent 108).

The user may also articulate and manipulate the endoscope to apply radial pressure urging the patch 106 into contact with the tissue and may even articulate the endoscope to add to apply the patch 106 as the stent 108 is slowly expanded. The user would then move the expansion device so that the patch 106 and the expansion device are rolled over the surface of the target portion of tissue to be treated. That is, the user would move the expansion device in a direction perpendicular to a longitudinal axis L of the expansion device while maintaining contact with the tissue so that, as the patch 106 adheres to the tissue, the patch 106 is unspooled from the expansion device in a direction A that is opposite of a direction B in which the patch 106 was rolled onto the expansion device. (See FIG. 10a). In this manner, the user may unspool a length of patch 106 that is greater than a diameter of the expanded expansion member to treat larger portions of target tissue. That is, if the patch 106 is wrapped around the stent 3 times, the user may unspool this entire length of patch 106 to treat a portion of tissue much larger than the surface area of the expanded expansion member.

As would be understood by those skilled in the art, this technique may be used with any of the described expansion devices. The stent 108 applies line contact forcing the patch 106 radially outward against the target tissue. As the stent 108 expands, the radial pressure on the patch 106 perpendicular to the longitudinal axis of the stent 108 unwraps the patch 106. In such a use, the stent 108 may be help stationary while the user manipulates the endoscope to unwrap the patch 106 from the stent 108 as each portion of the patch 106 becomes attached to tissue.

It will also be understood by those skilled in the art that the patch 106 (or plurality of patches 106) is formed to a desired length that is related to a length of the stent 108 when it is longitudinally extended so that, as the stent 108 is longitudinally compressed and radially expanded, the length of the longitudinally compressed stent 108 is sufficient to press a target portion of the patch 106 against the target tissue as desired. That is, depending on several factors including, for example, a stiffness of the patch 106, portions of the patch 106 extending longitudinally beyond a length of the stent 108 (in its radially extended state) will radially expand even though they are not being pressed outward from within. That is, for patches 106 of a certain stiffness, portions of the patch 106 not resting on the outer surface of the radially expanded stent 108 will expand outward when those portions of the patch 106 that are in contact with the stent 108 are pressed outward as the stiffness of the patch 106 allows these extended portions to cantilever outward from the supported portions of the patch 106 to press against the target tissue.

As would be understood by those skilled in the art, the length at which these cantilevered portions of the patch 106 will be properly adhered to the target tissue will depend on the stiffness of the patch 106, the properties of the adhesive material in the patch 106, properties of the target tissue, etc. If desired, after a first portion of a patch has been adhered to a first portion of tissue, the user may longitudinally extend and radially retract the stent 108, and move the radially contracted stent 108 to a new position inside the expanded patch 106. The user may then re-expand the stent 108 to press a new portion of the patch 106 into contact with a second portion of tissue. The user may repeat this procedure as often as needed to achieve the desired adherence between the entire patch 106 and the underlying tissue.

Those skilled in the art will understand that various modifications may be made to the disclosed embodiments without departing from the scope of this disclosure which is intended to be limited only by the scope of the claims appended hereto. For example, any of the various components of the several embodiments may be combined in any manner not specifically disclaimed in this disclosure.

1-10. (canceled)

11. A device for treating tissue, comprising:
a hollow sheath having sufficient flexibility to be inserted to a target site within a living body within a flexible endoscope, the sheath having a length selected to extend from a distal end which, when the device is in an operative configuration, is adjacent to the target site, to a proximal end which remains outside the living body accessible to a user;
a flexible hollow member extending through the sheath from a proximal end that remains outside the living body to a distal end which may be extended distally out of the sheath to contact a target portion of tissue adjacent to the target site;
an expansion device coupled to and extending distally from a distal end of the hollow member, the expansion device being movable from a radially compressed insertion configuration to a radially expanded deployed configuration; and
a first therapeutic patch wrapped around the expansion device, the first patch being configured to be forced radially outward into contact with and adhere to a first target portion of tissue when the expansion device is moved to the deployed configuration, the expansion device being configured to be moved, after the first patch has been adhered to the first target portion of tissue as desired, from the deployed configuration to the insertion configuration to separate the first patch from the expansion device leaving the first patch in a desired position adhered to the first target portion of tissue.

12. The device of claim 11, wherein the expansion device comprises a plurality of members extending from a proximal end of the expansion device to a distal end of the expansion device and wherein the device further comprising:
a control wire coupled to a distal end of the expansion device, movement of the control wire proximally relative to the expansion device compressing the expansion device along a longitudinal axis thereof and expanding the expansion device radially away from the longitudinal axis.

13. The device of claim 12, wherein the members of the expansion device are formed as an expandable tubular stent.

14. The device of claim 12, wherein a first member of the expansion device includes a proximal flexible section coupled to the hollow member, a rigid central member extending distally from the proximal flexible section and a distal flexible section coupled to a distal end of the control wire, the control wire extending proximally through the expansion device and through the hollow member to a proximal end that remains outside the living body manipulable by a user of the device.

15. The device of claim 14, further comprising:
a control member coupled to a slider of a handle of the device and wherein a proximal end of the hollow member is coupled to the handle, the control member extending through the hollow member and the expansion device to couple to a distal end of the expansion device.

16. The device of claim 14, wherein the proximal and distal flexible sections of the first member include one of Nitinol, of Nitional cobalt alloy wire, pebax, nylon, ptfe and copper.

17. The device of claim 14, wherein the rigid central member includes one of stainless steel, hypotube, a biocompatible hard plastic.

18. The device of claim 11, further comprising:
a second therapeutic patch wrapped around the expansion device and radially outside the first therapeutic patch, the second patch being configured to be forced radially outward into contact with and adhere to a second target portion of tissue when the expansion device is moved to the deployed configuration, the expansion device being configured to be moved, after the second patch has been adhered to the second target portion of tissue as desired, from the deployed configuration to the insertion configuration to separate the second patch from the expansion device leaving the second patch in a desired position adhered to the second target portion of tissue.

19. The device of claim 11, wherein the first therapeutic patch has a width extending along a longitudinal axis of the expansion device and a length transverse to the width, the first therapeutic patch being wrapped around the expansion device so that the length of the first therapeutic patch extends circumferentially around the expansion device, the length of the first therapeutic patch being configured to be at least as great as a diameter of the expansion device in the deployed configuration.

20. The device of claim 11, wherein the first therapeutic patch includes an adhesive having a predetermined cure time and wherein the first therapeutic patch is configured to be removable from tissue against which it has been pressed before the predetermined cure time has elapsed since the first therapeutic patch was pressed against the tissue.

21. A method for treating tissue, comprising:
inserting into a living body via a working channel of a flexible endoscope a device including a hollow sheath having received therein an expansion device with a first therapeutic patch wrapped around the expansion device, wherein the sheath is maintained during insertion in an insertion configuration in which the expansion device and the first therapeutic patch are maintained within the hollow sheath;
extending the sheath distally out of the endoscope to a target site adjacent to target tissue to be treated;
moving the sheath relative to the expansion device and the first therapeutic patch to expose the expansion device and the first therapeutic patch distally of a distal end of the sheath;
expanding the expansion device to move the first therapeutic patch radially outward into contact with a first target portion of tissue to be treated; and
radially contracting the expansion device out of contact with the first therapeutic patch to leave the first therapeutic patch in a desired position adhered to the first target portion of tissue.

22. The method of claim 21, wherein the first therapeutic patch has a width extending along a longitudinal axis of the expansion device and a length transverse to the width, the first therapeutic patch being wrapped around the expansion device so that the length of the first therapeutic patch extends circumferentially around the expansion device, the length of the first therapeutic patch being configured to be at least as great as a diameter of the expansion device in the deployed configuration, further comprising the step of:
pressing the first therapeutic patch against the first target portion of tissue and moving the expansion device relative to the first target portion of tissue in a direction corresponding to the length of the first therapeutic patch to unroll the first therapeutic patch from the expansion device over the first target portion of tissue until the entire length of the first therapeutic patch has been adhered to the first target portion of tissue.

23. The method of claim 21, wherein the device further includes a second a therapeutic patch wrapped around the first therapeutic patch and the expansion device.

24. The method of claim 21, wherein the expansion device is a radially expandable stent and therein the device includes a mechanism for longitudinally compressing the stent to radially expand the stent.

25. The method of claim 21, wherein the expansion device is coupled to a distal end of a hollow member that extends through the sheath to a proximal end that remains outside the living body, the expansion device including a plurality of longitudinal members, each of the longitudinal members including a proximal flexible section coupled to the hollow member, a rigid central member extending distally from the proximal flexible section and a distal flexible section, further comprising the step of, manipulating a control wire that extends through the hollow member and the expansion device to couple to the distal flexible sections of the longitudinal members to longitudinally compress and radially expand the expansion device.
